(19) United States
(12) Patent Application Publication
(10) Pub. No.: US 2025/0248808 A1
(43) Pub. Date: Aug. 7, 2025

(54) VASCULAR STENTS AND SUPPORT STRUCTURES FOR PROSTHETIC HEART VALVES

(71) Applicant: EDWARDS LIFESCIENCES CORPORATION, Irvine, CA (US)

(72) Inventor: John J. Desrosiers, San Clemente, CA (US)

(73) Assignee: EDWARDS LIFESCIENCES CORPORATION, Irvine, CA (US)

(21) Appl. No.: 19/188,670

(22) Filed: Apr. 24, 2025

Related U.S. Application Data
(63) Continuation of application No. PCT/US2023/079788, filed on Nov. 15, 2023.
(60) Provisional application No. 63/384,275, filed on Nov. 18, 2022.

(51) Int. Cl. A61F 2/24

(52) U.S. Cl. CPC A61F 2/2418; A61F 2/2436; A61F 2220/0008

(57) ABSTRACT

A radially expandable frame includes a main body and a plurality of axially extending retention members extending axially from the stent. The frame can be deployed adjacent to a native annulus of a patient, with the retention members extending through the native annulus to retain the native leaflets in an open configuration. The retention members can also space the stent apart from the native valve in an upstream or downstream direction. The frame may be deployed as a docking station for a prosthetic heart valve or may have a valvular structure attached directly to the frame. Also disclosed herein are methods for deploying the radially expandable frame in a patient. The methods include methods for recapturing the frame into a delivery apparatus by the retention members.

### CROSS REFERENCE TO RELATED APPLICATIONS

This application is a continuation of International Application No. PCT/US2023/079788, filed Nov. 15, 2023, which claims the benefit of U.S. Provisional Application No. 63/384,275, filed Nov. 18, 2022, which is incorporated by reference herein in its entirety.

### FIELD

The present disclosure relates to vascular stents and support structures for prosthetic heart valves, as well as methods for implanting the same.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size. The prosthetic heart valve can include valve structure (e.g., leaflets) for regulating blood flow in a single direction. A vascular stent can be compressed and delivered in a similar way to a desired location within a patient's vasculature. A stent, however, does not include a valve structure. In some instances, a prosthetic heart valve can be deployed within a stent such that the stent serves as a support structure or an anchoring mechanism for the prosthetic heart valve.

### SUMMARY

Described herein are prosthetic heart valves, delivery apparatus, and methods for implanting prosthetic heart valves. The disclosed prosthetic heart valves, delivery apparatus, and methods can, for example, provide improved control over placement of the location of the valve deployment relative to the native heart valve. The disclosed prosthetic heart valves can also allow for improved ability to recapture the valve during the implantation process. As such, the devices and methods disclosed herein can, among other things, overcome one or more of the deficiencies of typical prosthetic heart valves and their delivery apparatus. Stents and related methods are also disclosed herein.

A prosthetic heart valve includes a frame (which can also be referred to as “a stent” or “a support structure”) and a valve structure (e.g., leaflets) configured for regulating the flow of blood in one direction. In addition to these components, a prosthetic heart valve can comprise one or more of the components disclosed herein.

In some examples, a prosthetic heart valve can include a paravalvular leakage (PVL) skirt (which can also be referred to as a sealing member) coupled to the frame.

A stent or support structure for a prosthetic heart valve (which can also be referred to as “a docking station”) can comprise a plurality of struts that can be radially compressed and radially expanded.

In some examples, a prosthetic heart valve can comprise one or more retention members extending axially from an end portion of the support structure.

In some examples, a prosthetic heart valve can be partially deployed from a delivery apparatus by radially expanding the support structure while retaining the one or more retention members in the delivery apparatus.

In some examples, a partially deployed prosthetic heart valve can be recaptured into a delivery apparatus via one or more retention members retained in the delivery apparatus.

In some examples, a prosthetic heart valve can comprise a support structure comprising a plurality of major and minor cells arranged in rows of major cells and minor cells.

In some examples, a prosthetic heart valve can comprise a lesser number of cell apices at a first end portion of the support structure than at a second end portion of the support structure.

In some examples, a prosthetic heart valve can comprise a retention member extending from each cell apex along a first end portion of the support structure.

In some examples, a prosthetic heart valve comprises one or more of the components recited in Examples 1-103 below.

Certain examples concern a prosthetic heart valve, comprising a radially expandable main body having a first end portion, a second end portion, and a plurality of interconnected struts. The interconnected struts extend from the first end portion to the second end portion and define a plurality of cells arranged in rows of cells. A valvular structure is disposed within and attached to the radially expandable main body and configured to regulate a flow of blood through the radially expandable main body in one direction. A plurality of retention members extends axially from the first end portion of the radially expandable main body. The plurality of cells comprises a plurality of major cells and a plurality of minor cells, the minor cells being smaller than the major cells. A row of cells nearest the first end portion of the radially expandable main body comprises major cells, and the retention members extend axially from the major cells.

Certain examples concern an expandable frame. The expandable frame comprises a main body having a first end portion, a second end portion, and a longitudinal axis extending between the first end portion, and a plurality of interconnected struts extending from the first end portion to the second end portion along the longitudinal axis. The plurality of interconnected struts forms a plurality of cells. The expandable frame also comprises a plurality of retention members extending longitudinally from the first end portion. The plurality of retention members is configured to extend through an annulus of a native heart valve and to retain one or more native leaflets of the native heart valve in an open state. The main body is configured to receive a prosthetic heart valve and secure the prosthetic heart valve at a location spaced apart longitudinally from the native heart valve.

Certain examples concern a medical assembly. The medical assembly comprises a first frame having a main body with a first end portion, a second end portion, and a longitudinal axis extending between the first end portion and the second end portion. The first frame also comprises a plurality of interconnected struts extending from the first end portion to the second end portion and forming a plurality of cells and a plurality of retention members extending longitudinally from the first end portion of the main body. The medical assembly also comprises a prosthetic heart valve disposed within and secured to the main body. The prosthetic heart valve comprises radially expandable second frame and a valvular structure disposed within the radially expandable frame and configured to regulate a flow of blood through the prosthetic heart valve. The plurality of retention members is configured to extend through an annulus of a native heart valve and to retain one or more native leaflets of the native heart valve in an open state. The main body is configured to secure the prosthetic heart valve at a location spaced apart longitudinally from the native heart valve.

Certain examples concern a method comprising advancing a delivery apparatus comprising an outer shaft containing a prosthetic heart valve, the prosthetic heart valve comprising a frame having a main body and a plurality of retention members extending axially from the main body, in a distal direction through a vasculature of a patient to a native annulus. The method also comprises partially deploying the prosthetic heart valve from the delivery apparatus adjacent to a native heart valve of the patient, wherein when the prosthetic heart valve is partially deployed, a valvular structure is engaged with a flow of blood and the retention members are attached to the delivery apparatus. The method also comprises advancing or retracting the deployed prosthetic heart valve in a direction defined by the flow of blood through the native heart valve until the retention members extend through a native annulus of the native heart valve. The method also comprises detaching the prosthetic heart valve from the delivery apparatus and expanding the retention members to a fully expanded state. When the retention members are in the fully expanded state, the retention members contact one or more native leaflets of the native heart valve and retain the one or more native leaflets in an open state. The prosthetic heart valve further comprises a valvular structure disposed within the frame and configured to regulate the flow of blood through the frame. The prosthetic heart valve is secured to the delivery apparatus by the plurality of axially extending retention members. The valvular structure is axially spaced apart from the native annulus of the native heart valve in the direction defined by the flow of blood.

Certain examples concern a method comprising advancing a delivery apparatus comprising an outer shaft containing a radially expandable frame, the radially expandable frame comprising a main body and a plurality of retention members extending axially from the main body, through a vasculature of a patient to a native annulus. The method also comprises partially deploying the radially expandable frame from the delivery apparatus adjacent to a native heart valve of the patient and positioning the radially expandable frame relative to the native heart valve such that the retention members extend through a native annulus of the native heart valve. The method also comprises fully deploying the radially expandable frame from the delivery apparatus and expanding the radially expandable frame to a fully expanded state, wherein an outer diameter of the main body contacts the vasculature of the patient and the retention members contact one or more native leaflets of the native heart valve and retain the one or more native leaflets in an open state. When the radially expandable frame is fully radially expanded, the main body is axially spaced apart from the native heart valve. The radially expandable frame is configured to receive a prosthetic heart valve and secure the prosthetic heart valve relative to the native heart valve.

Certain examples concern a method comprising advancing a delivery apparatus comprising an outer shaft containing a radially expandable frame, the radially expandable frame comprising a main body and a plurality of retention members extending axially from the main body, through a vasculature of a patient to a native annulus. The method also comprises partially deploying the radially expandable frame from the delivery apparatus adjacent to a native heart valve of the patient. The method also comprises recapturing the radially expandable frame within the delivery apparatus and adjusting the position of the radially expandable frame relative to the native heart valve of the patient. The method also comprises fully deploying the radially expandable frame from the delivery apparatus such that the radially expandable frame engages the vasculature of the patient.

Certain examples concern a method comprising sterilizing the medical assembly, the radially expandable frame, the main body, or the prosthetic heart valve of any preceding claim.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is an elevation view of a portion of a frame of a docking station in a radially-expanded state.

FIG. 2 is a perspective view of the frame of FIG. 1 in a radially-compressed state.

FIG. 3 is a perspective view of a docking station including the frame of FIG. 1.

FIG. 4 is a cut-away view of the docking station of FIG. 3 deployed at an implantation location within a patient's anatomy, which is depicted schematically in cross-section, and with a prosthetic heart valve deployed therein.

FIG. 5A is a perspective view of a delivery apparatus for deploying a docking station.

FIG. 5B illustrates the docking station of FIG. 3 disposed around a distal portion of the delivery apparatus of FIG. 5A.

FIG. 6A is an elevation view of a distal portion of the delivery apparatus of FIG. 5A with an outer shaft of the delivery apparatus in a retracted position.

FIG. 6B is an elevation view of a distal portion of the delivery apparatus of FIG. 5A with an outer shaft of the delivery apparatus in an extended position and cut away to show an encapsulated docking station.

FIGS. 6C-6F illustrate stages in deployment of the docking station of FIG. 3 from the delivery apparatus of FIG. 5A.

FIG. 7 illustrates a frame according to one example, depicted in a radially expanded state in a pulmonary artery.

FIG. 8A is a frame according to another example having axially extending retention members, in a radially expanded state.

FIG. 8B is a perspective view of the frame of FIG. 8A.

FIG. 8C is a second end view of the frame of FIG. 8A.

FIG. 8D is a first end view of the frame of FIG. 8A.

FIG. 9 illustrates the radially expandable frame of FIG. 8A deployed in the pulmonary artery.

FIGS. 10A-10D illustrate the frame of FIG. 8A in various stages of deployment from the delivery apparatus of FIG. 5A.

FIG. 11 is an end view of a prosthetic heart valve comprising the frame of FIG. 8A and a valvular structure attached thereto.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, the terms “upstream direction” and “downstream direction” refer to a direction relative to the flow of blood in a patient's vasculature. Thus, for example, when a device is upstream of a native valve, blood passes through or past the device to reach the native valve. When a device is downstream of a native valve, blood passes through or past the native valve to reach the device.

As used herein, the terms “within” and “between” when used in relation to a numerical range includes the numerical endpoints of that range, unless otherwise specified. For example, “within a range of 10% to 150%” indicates a numerical range including 10%, 150%, and every percentage value between 10% and 150%.

### Introduction to the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

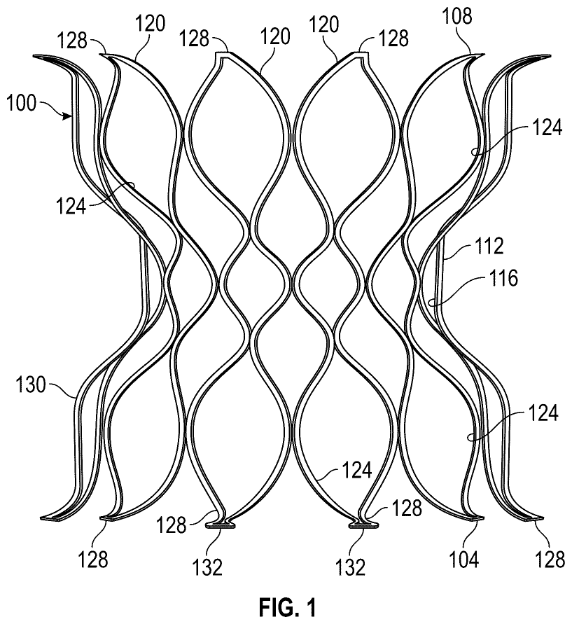

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO 2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

### The Disclosed Technology and Exemplary Examples

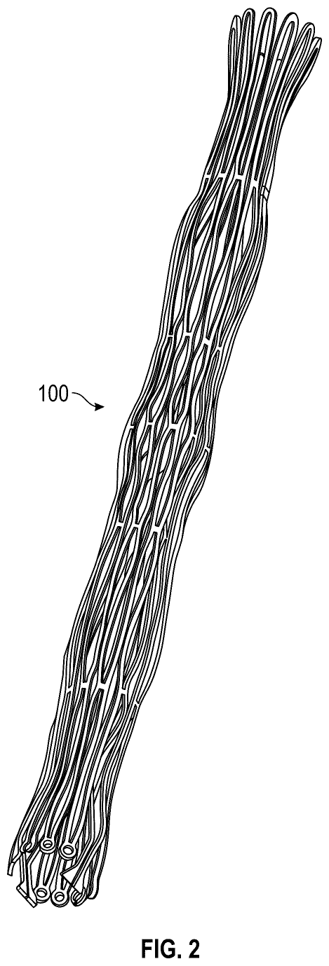

Turning now to the drawings, FIG. 1 illustrates an exemplary implementation of a frame 100 (or stent) that can form a body of a docking station. The frame 100 has a first end 104 and a second end 108. In some examples, the first end 104 can be an inflow end, and the second end 108 can be an outflow end. In other examples, the first end 104 can be an outflow end, and the second end 108 can be an inflow end. The terms “inflow” and “outflow” are related to the normal direction of blood flow (e.g., antegrade blood flow) through the frame. In the unconstrained, expanded state of the frame 100 shown in FIG. 1, a relatively narrower portion (or waist) 112 of the frame 100 between the first end 104 and the second end 108 forms a valve seat 116. The frame 100 can be compressed (as illustrated in FIG. 2) for delivery to an implantation location by a delivery apparatus.

Although the docking stations, delivery apparatus, prosthetic heart valves, and/or methods are described herein with respect to a particular implantation location (e.g., a pulmonary valve) and/or a particular delivery approach (e.g., transfemoral), the device and methods disclosed herein can be adapted to various other implantation locations (e.g., an aortic valve, a mitral valve, and a tricuspid valve) and/or delivery approaches (e.g., transapical, transseptal, etc.).

In the example illustrated by FIG. 1, the frame 100 includes a plurality of interconnected struts 120 arranged to form cells 124. The ends of the struts 120 can form apices 128 at the ends of the frame 100 at the intersection of two struts 120. One or more of the apices 128 can include a connector tab 132. The portions of the struts 120 between the apices 128 and the valve seat 116 (or the waist 112) form a sealing member 130 of the frame 100. In the unconstrained, expanded state of the frame 100 illustrated in FIG. 1, the apices 128 extend generally radially outward and are radially outward of the valve seat 116.

The frame 100 can be made of a highly resilient or compliant material to accommodate large variations in the anatomy. For example, the frame 100 can be made of a flexible metal, metal alloy, polymer, or an open cell foam. An example of a highly resilient metal is Nitinol, which is a metal alloy of nickel and titanium, but other metals and high resilient or compliant non-metal materials can be used. The frame 100 can be self-expanding, manually expandable (e.g., expandable via a balloon), or mechanically expandable. A self-expanding frame can be made of a shape memory material, such as, for example, Nitinol. In this manner, the frame can be radially compressed as depicted in FIG. 2 (e.g., via a crimping device) and can radially expand to the configuration depicted in FIG. 1.

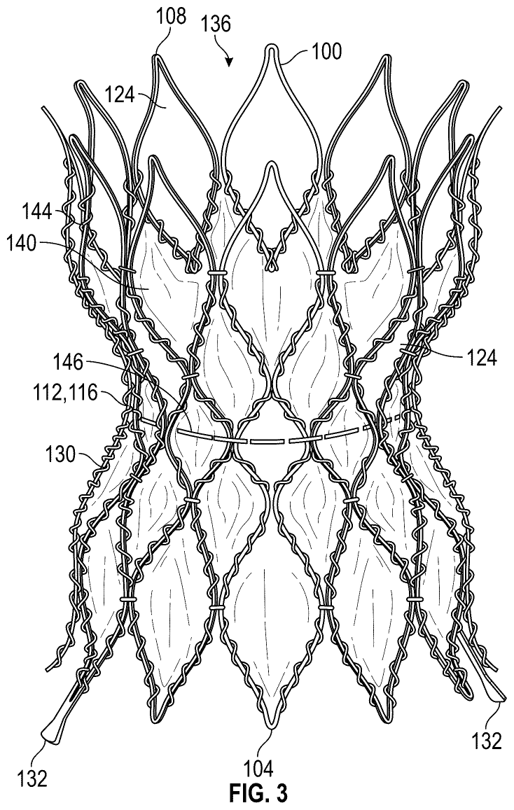

FIG. 3 illustrates an exemplary docking station 136 including the frame 100 and an impermeable material 140 disposed within the frame. The impermeable material 140 is attached to the frame 100 (e.g., by sutures 144). In the example illustrated by FIG. 3, the impermeable material 140 covers at least the cells 124 in the sealing member 130 of the frame 100. The seal formed by the impermeable material 140 at the sealing member 130 can help funnel blood flowing into the docking station 136 from the proximal inflow end 104 to the valve seat 116 (and the valve once installed in the valve seat). One or more rows of cells 124 proximate to the distal outflow end 108 can be open.

The impermeable material 140 can be a fabric that is impermeable to blood. A variety of biocompatible materials can be used as the impermeable material 140, such as, for example, foam or a fabric that is treated with a coating that is impermeable to blood, a polyester material, or a processed biological material, such as pericardium. In one particular example, the impermeable material 140 can be polyethylene terephthalate (PET).

The docking station 136 may include a band 146 that extends around the waist 112 (or that is integral to the waist) of the frame 100. The band 146 can constrain expansion of the valve seat 116 to a specific diameter in the deployed state to enable the valve seat 116 to support a specific valve size. The band 146 can take on a wide variety of different forms and can be made of a wide variety of different materials. For example, the band 146 can be made of PET, one or more sutures, fabric, metal, polymer, a biocompatible tape, or other relatively nonexpanding materials known in the art and that can maintain the shape of the valve seat 116.

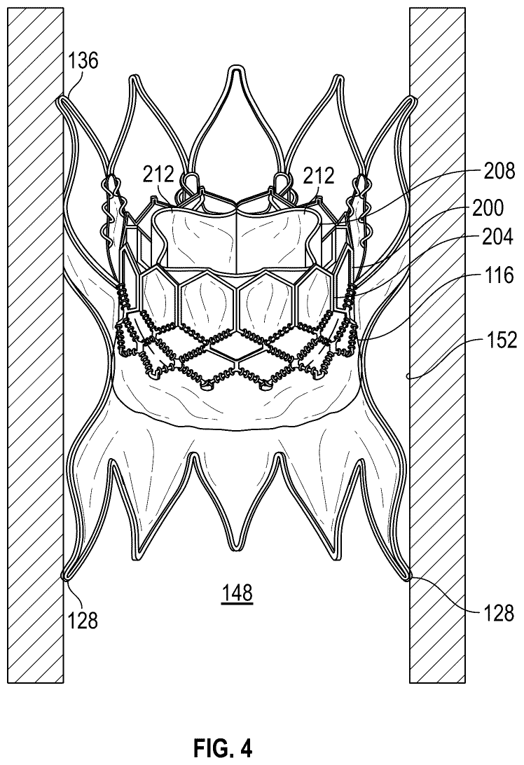

FIG. 4 illustrates the docking station 136 in a deployed state within a native valve annulus 148. As can be seen, the frame 100 of the docking station 136 is in an expanded condition, with the end portions of the frame pressed against the inner surface 152 of the native valve annulus. The band 146 (shown in FIG. 3) can maintain the valve seat 116 at a constant or substantially constant diameter in the expanded condition of the frame 100. FIG. 4 also shows a prosthetic valve 200 deployed within the docking station 136 and engaged with the valve seat 116 of the docking station 136. The prosthetic valve 200 can be implanted by first deploying the docking station 136 at the implantation location and then installing the prosthetic valve within the docking station.

The prosthetic valve 200 can be configured to replace a native heart valve (e.g., aortic, mitral, pulmonary, and/or tricuspid valves). In one example, the prosthetic valve 200 can include a frame 204 and a valvular structure 208 disposed within and attached to the frame 204. The valvular structure 208 can include one or more leaflets 212 that cycle between open and closed states during the diastolic and systolic phases of the heart. The frame 204 can be made of the frame materials described for the frame 100 of the docking station 136. The leaflets 212 can be made in whole or in part from pericardial tissue (e.g., bovine pericardial tissue), biocompatible synthetic materials, or various other suitable natural or synthetic materials known in the art.

The docking station 136 is not limited to use with the particular example of the prosthetic valve 200 illustrated in FIG. 4. For example, mechanically expandable prosthetic valves such as described in U.S. Patent Publication Nos. 2018/0153689 and 2019/0060057; International Publication Nos. WO 2021/003167 and WO 2020/081893, which are incorporated by reference herein, may be installed in the docking station 136.

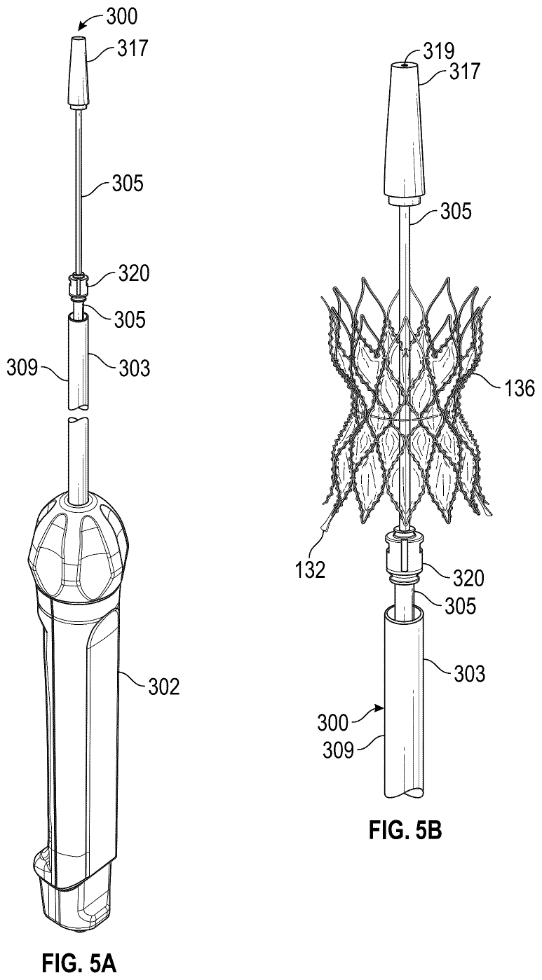

FIG. 5A illustrates an exemplary delivery apparatus 300 that can be used to deliver the docking station to an implantation location. The delivery apparatus 300 generally includes a handle 302 and a shaft assembly 303 coupled to the handle 302 and extending distally from the handle 302. The shaft assembly 303 includes an inner shaft 305 and an outer shaft 309. The inner shaft 305 extends through a lumen of the outer shaft 309.

In the example illustrated by FIG. 5A, a frame connector 320 is coupled to the inner shaft 305. The docking station 136 can be disposed around a portion of the inner shaft 305 extending distally from the frame connector 320, as shown in FIG. 5B. In one example, the frame connector 320 includes one or more recesses 322 that can receive one or more connector tabs 132 at the proximal end of the docking station 136 and thereby axially restrain the docking station 136.

A nosecone 317 can be attached to a distal end of the inner shaft 305. The nosecone 317 includes a central opening 319 for receiving a guidewire. As such, a proximal end of the guidewire can be inserted into the central opening 319 and through the inner shaft 305, and a distal end portion of the delivery apparatus 300 can be advanced over the guidewire through a patient's vasculature and to an implantation location. The guidewire can pass through the nosecone 317 into the inner shaft 305 during advancing of the delivery apparatus through a patient's vasculature.

The handle 302 can be operated to move the outer shaft 309 relative to the inner shaft 305, generally between an extended position and a retracted position. The handle 302 can be extended to slide the outer shaft 309 over the frame connector 320 and over any docking station coupled to the frame connector 320 to encapsulate the docking station within the outer shaft 309. As the outer shaft 309 slides over the docking station 136, the outer shaft 309 can compress the docking station 136 such that the docking station is encapsulated within the outer shaft 309 in the compressed state. In the fully extended position, a distal end of the outer shaft 309 can abut a proximal end of the nosecone 317 such that there are no gaps in the delivery assembly. Additionally, or alternatively, a crimping device can be used to radially compress the docking station such that it can be inserted into the outer shaft of the delivery apparatus.

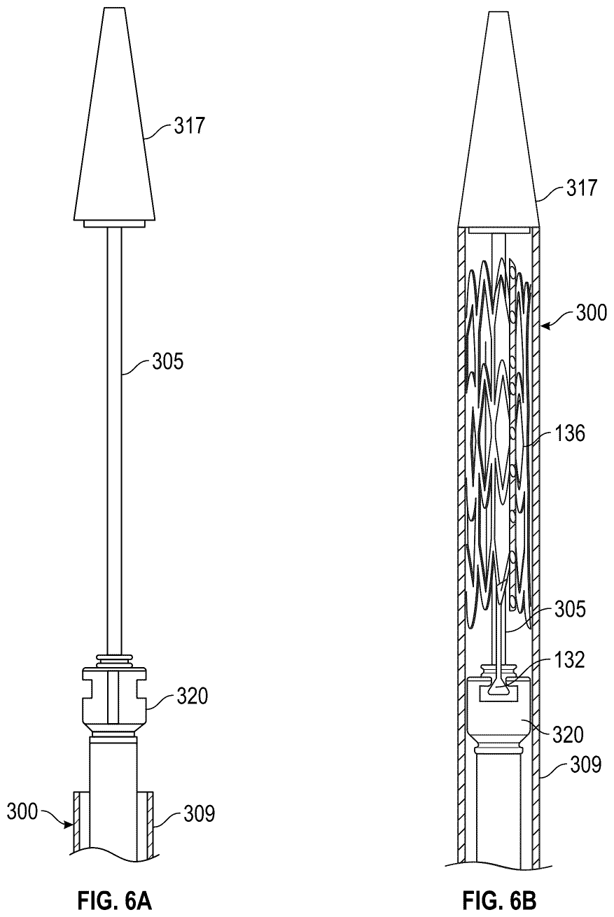

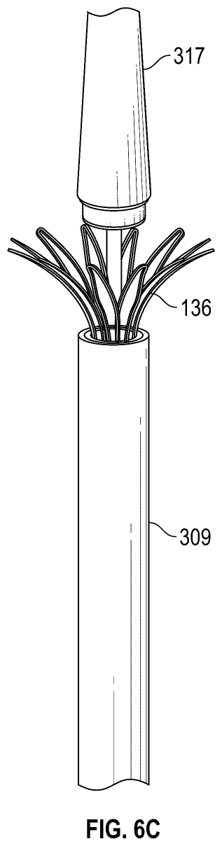

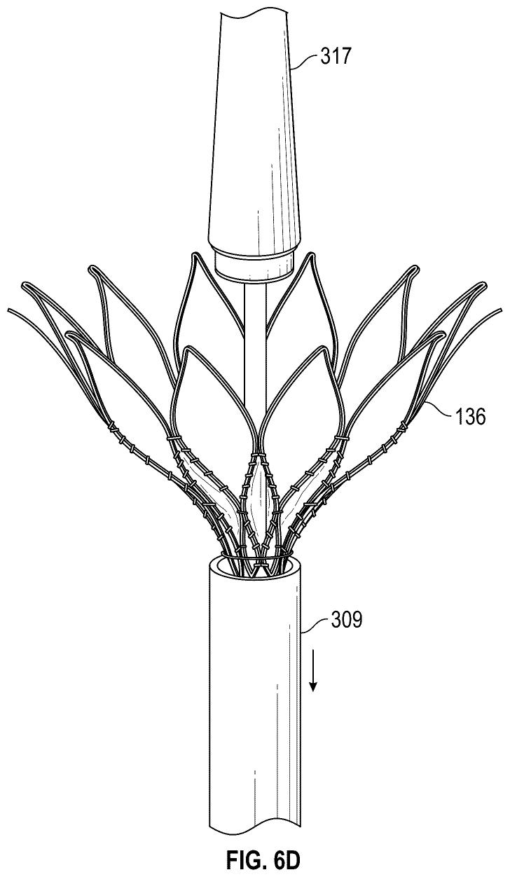

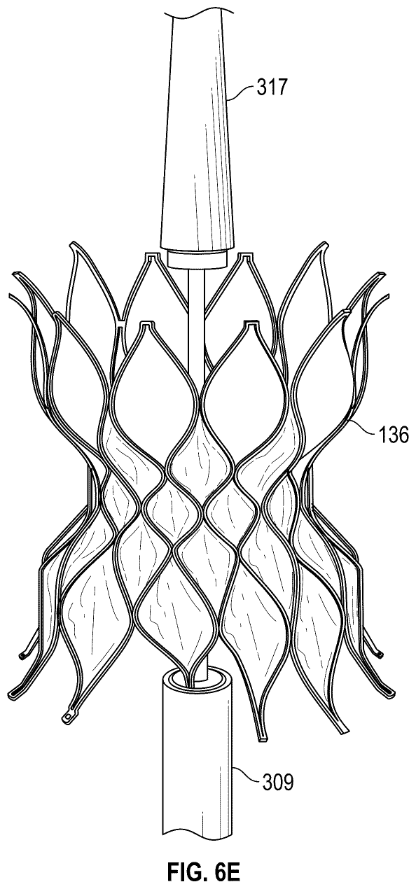

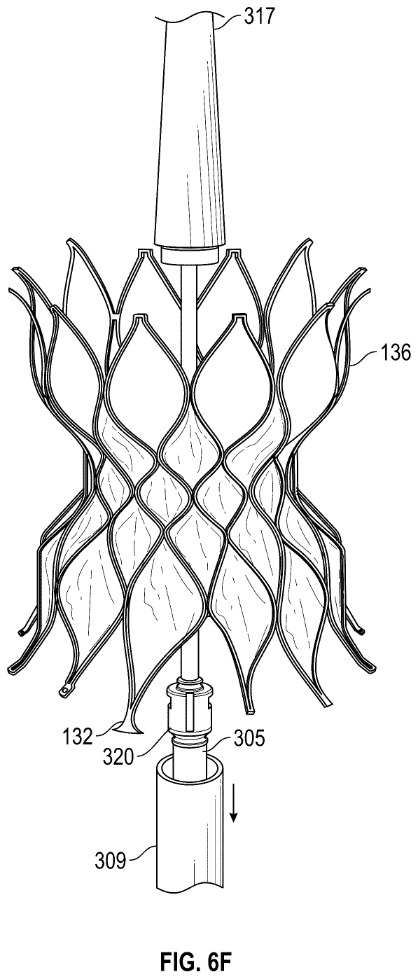

FIGS. 6A-6F illustrate a method of deploying a docking station at an implantation location within an anatomy. For purposes of illustration, the patient's anatomy is omitted. As shown in FIG. 6A, the method includes retracting the outer shaft 309 by the handle of the delivery apparatus to allow loading of the docking station 136 onto the inner shaft 305. FIG. 6B depicts disposing the docking station 136 around the inner shaft 305 and engaging each of the connector tabs 132 of the docking station 136 with the frame connector 320. The outer shaft 309 is then positioned over the docking station 136 such that the docking station 136 is encapsulated therein. This can be accomplished by manipulating the handle of the delivery apparatus. As shown in FIG. 6B, the distal end of the outer shaft 309 abuts the proximal end of the nosecone 317.

The method further includes inserting the delivery apparatus, from the nosecone 317 end, into a patient's vasculature and advancing the delivery apparatus through the patient's vasculature to the implantation location.

At the implantation location, the method includes retracting the outer shaft 309 by the handle of the delivery apparatus to expose the docking station 136. FIGS. 6C-6F show different stages of retracting the outer shaft 309. As can be seen, in cases where the docking station 136 is self-expanding, the docking station 136 gradually emerges from the outer shaft 309 and gradually expands from the compressed state as the outer shaft 309 is retracted. When the outer shaft 309 is sufficiently retracted, the connector tabs 132 disengage from the frame connector 320. Once the docking station 136 is disengaged from the frame connector 320, the docking station 136 can radially expand to engage the anatomy.

When the docking station 136 is a self-expanding docking station where the docking station 136 and the connector tabs 132 are naturally biased toward an expanded configuration. While the docking station 136 is attached to the delivery system, the docking station 136 is compressed to a smaller configuration (shown in FIG. 6B) for insertion and tracking through the vasculature. The compressed configuration of the docking station is held in place axially by the frame connector 320 (which is fixed relative to the inner shaft 305) and held in place radially by the outer shaft 309. The docking station 136 is therefore prevented from premature deployment by the frame connector 320 and the outer shaft 309. Once the docking station 136 is at the implantation location within the anatomy, the outer shaft 309 can be retracted to expose and deploy the docking station 136.

As the outer shaft 309 is retracted to expose the docking station 136, the distal portion of the docking station 136 expands (as shown, for example, in FIGS. 6C and 6D). In some cases, prior to completing retraction of the outer shaft 309, it may be desirable to reposition and/or retrieve the docking station 136. In this case, the outer shaft 309 may be extended again to recapture and recompress the docking station 136 in order to allow the docking station 136 to be repositioned and/or retrieved. However, the bias toward an expanded configuration can create an axial tension between the docking station and the frame connector. The axial tension can concentrate at the flanges of the connector tabs of the docking station as the outer shaft is extended distally over the docking station for recapture. Due to the relatively high forces during recapture and/or retrieval, the connector tabs of the docking station tend to move radially outwardly attempting to disengage from the frame connector 320. This can increase the force required to recapture the docking station. In extreme instances, the connector tabs can disengage from the connector, which can inhibit recompression and/or retrieval of the docking station.

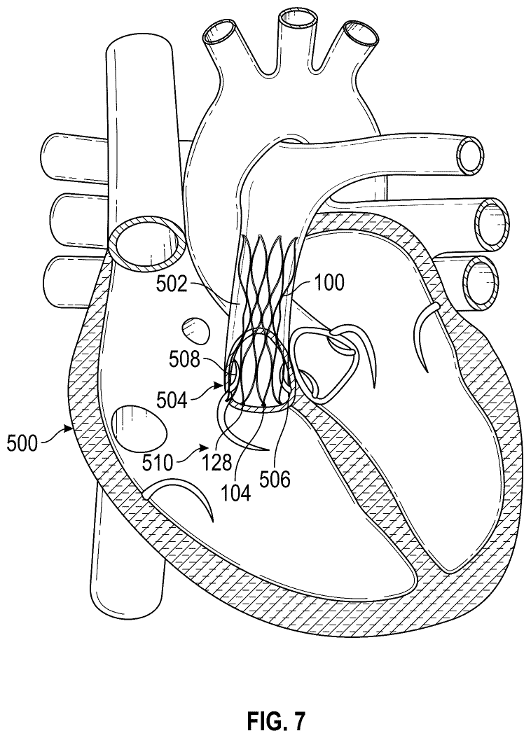

In some cases, such as that shown in FIG. 7, the frame 100 can be positioned within a heart 500 of the patient, such as within the pulmonary artery 502. More particularly, the frame 100 can be positioned within the native valve 504 such that one or more portions of the frame 100 (e.g., one of the end portions (104, 108) contacts one or more native leaflets 508 and restrains the native leaflets in an open configuration. In the illustrated example, the apices 128 of the inflow end 104 extend through the annulus 506 of the native heart valve 504 and pin the native leaflets 508 against the annulus 506 of the native valve 504 in an open configuration. This can, for example, prevent the native leaflets 508 from interfering with the flow of blood through a prosthetic heart valve (such as prosthetic heart valve 200 previously discussed) deployed within the frame 100 and/or a valve deployed downstream of the native valve.

In certain circumstances, it may be advantageous to position the docking station 136 such that a prosthetic heart valve (such as prosthetic heart valve 200) deployed therein is spaced apart from the native heart valve 148. Accordingly, in some examples, a docking frame and/or a prosthetic heart valve, such as those discussed in greater detail below, can include one or more retention members that extend axially from at least one end portion. The retention members can be configured to extend from a main body of the frame to the native leaflets to pin the native leaflets in an open state, and/or to space the main body of the docking stent and/or prosthetic heart valve apart from the native heart valve. In addition to providing greater variability in the deployment location, the retention members can, for example, also aid in the deployment procedure, as further explained below.

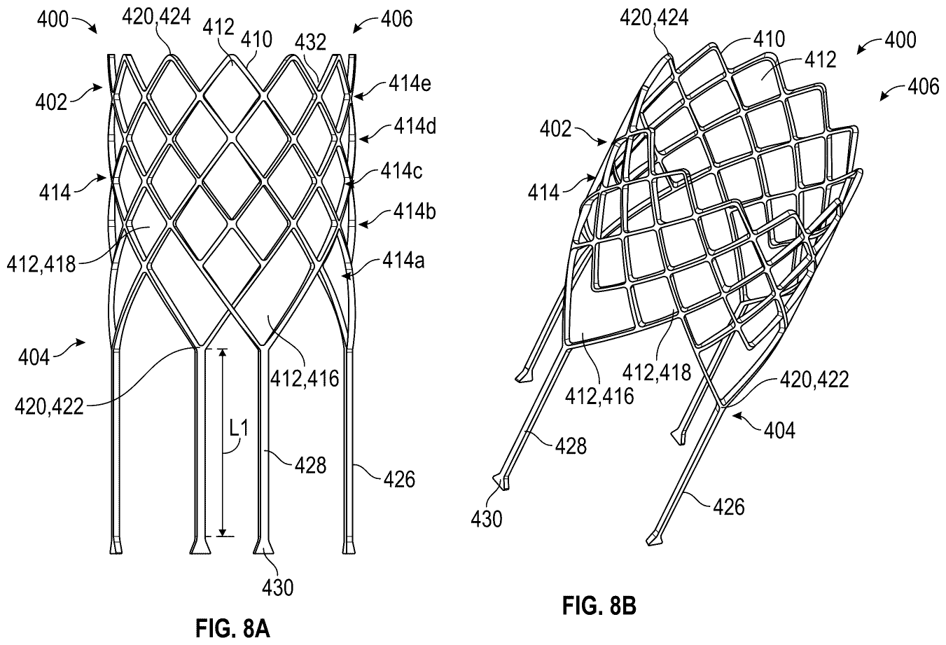

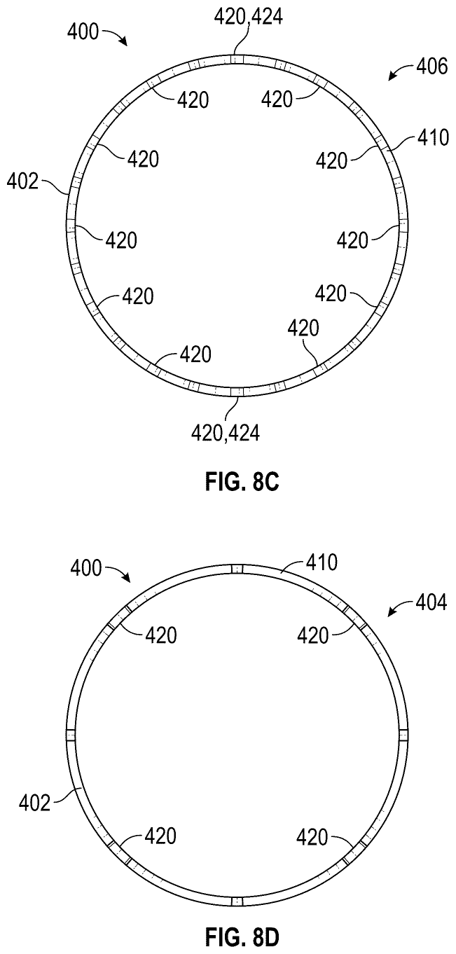

FIGS. 8A and 8B illustrate an example of a frame 400 with elongated retention members. In some examples, the retention members can be used to pin the leaflets of a native heart valve in an open state and/or to space the main body of the frame 400 apart from the native heart valve. The frame 400 can be deployed with any delivery system previously discussed, such as delivery system 300, and can receive a prosthetic heart valve deployed therein, such as prosthetic heart valve 200. The retention members can, in some instances, aid in the deployment procedure.

As shown in FIG. 8B, the frame 400 comprises a main body 402 and a plurality of retention members 426 extending from the main body 402. The main body 402 can secure a valvular structure, such as a prosthetic heart valve as disclosed herein within the vasculature of a patient. The retention members 426 can secure the native leaflets of the vasculature of the patient in an open configuration, and in some examples can allow for the main body 402 to be positioned away from a native annulus of the vasculature of the patient.

The main body 402 of the frame 400 comprises a first end portion 404 and a second end portion 406. In some examples, the first end portion 404 can be an inflow end portion, and the second end portion 406 can be an outflow end portion. In other examples, the first end portion 404 can be an outflow end portion, and the second end portion 406 can be an inflow end portion. The terms “inflow” and “outflow” are related to the normal direction of blood flow, as discussed above in relation to frame 100.

Turning now to FIG. 8A, the main body 402 comprises a plurality of interconnected struts 410 arranged to form cells 412. In some examples, the cells can be arranged in rows 414 of cells 412 extending between the first end portion 404 and the second end portion 406 of the main body 402. For instance, as illustrated in FIG. 8A, one example frame body can have a plurality of cells 412 arranged in 5 rows 414 of cells (for instance, a first row 414a, a second row 414b, a third row 414c, a fourth row 414d, and a fifth row 414e) between the first end portion 404 and the second end portion 406 of the main body 402. It is to be appreciated that other example frames 400 can include a shorter or longer main body 402 with a correspondingly lesser or greater number of rows of cells, such as 2, 3, 4, 6, 7, 8, 9, 10, 11, or 12 rows of cells, depending on the desired length of the frame body.

In some examples, such as the example illustrated in FIGS. 8A-8D, the cells 412 can further comprise a plurality of major cells 416 and minor cells 418. As shown in FIG. 8A, the major cells 416 can be larger than the minor cells 418, and may have a different geometry (for example, as shown in FIG. 8A, the major cells 416 can have a heart shaped geometry or teardrop shaped geometry, while the minor cells 418 have a diamond shaped geometry). It is to be understood, however, that the major cells 416 and minor cells 418 are not limited to any specific geometry, and rather may be adapted in shape and size to the needs of the frame 400.

In certain examples, the major cells 416 and minor cells 418 can be arranged in respective rows 414 of major cells 416 and minor cells 418. For instance, as shown in FIG. 8A, the first row 414a of cells 412 can be formed exclusively of major cells 416, and the remaining rows 414 of cells 412 (414b, 414c, 414d, and 414e in the illustrated example) can be formed exclusively of minor cells 418. While the frame 400 shown in FIGS. 8A-8D has the major cells 416 disposed in a row 414a of cells 412 along the first end portion 404 (that is, the inflow end portion in the illustrated example) of the main body 402, it is to be understood that in other examples, the row 414 of cells 412 nearest the second end portion 406 (that is, the outflow end portion in the illustrated example) can also comprise major cells 416. In other examples, the row 414 of cells 412 nearest both the first end portion 404 and the second end portion 406 can comprise major cells 416.

With continued reference to FIG. 8A, the ends of the struts 410 form apices 420 at the end portions 404, 406 of the main body 402. As shown, the apices 420 can further comprise a plurality of major apices 422, formed by the struts 410 forming the major cells 416, and a plurality of minor apices 424 formed by the struts 410 forming the minor cells 418. In examples of frame 400 having a row 414 of major cells 416 disposed at the first end 404 of the main body 402, and a row 414 of minor cells 418 disposed at the second end 406 of the main body 402, such as the example illustrated in FIGS. 8A-8D, the frame body may have different number of apices 420 at each end portion.

For example, as illustrated in FIG. 8B, the second end portion 406 of the main body 402 can comprise 12 apices 420, formed by the struts 410 defining the row 414 of cells 412 nearest the second end portion 406. It is to be appreciated that, while FIG. 8B depicts a second end portion 406 having 12 apices 420, in other examples, the second end portion 406 might have a lesser or greater number of apices 420, such as 6, 7, 8, 9, 10, 11, 13, 14, 15, 16, 17, or 18 apices, according to the desired size of the main body 402 and the cells 412. It is also to be appreciated that, when the row 414 of cells 412 nearest the second end portion 406 comprises one or more major cells 416, the second end portion 406 may comprise a lesser number of apices, such as 2-8 apices, or 2, 3, 4, 5, 6, 7, or 8 apices.

As illustrated in FIG. 8D, the first end portion 404 of the main body 402 can comprise four apices 420 formed by the struts 410 defining the row 414 of major cells 416 nearest the first end portion 404. It is to be appreciated that, while FIG. 8D depicts a first end portion of 404 having four apices 420, in other examples, the first end portion 404 might have a lesser or greater number of apices 420, such as 2, 3, 5, 6, 7, or 8 apices, according to the desired size of the main body 402 and the cells 412.

The frame 400 illustrated in FIGS. 8A-8D can further include paravalvular leakage (PVL) skirt (which can also be referred to as a sealing member) with the same or substantially the same basic configuration as the sealing member 130 of the frame 100 previously discussed and best illustrated in FIG. 3. The sealing portion can help funnel blood flowing into the main body 402 of the frame 400 and through a prosthetic heart valve or valvular structure affixed thereto, as previously described in relation to the frame 100.

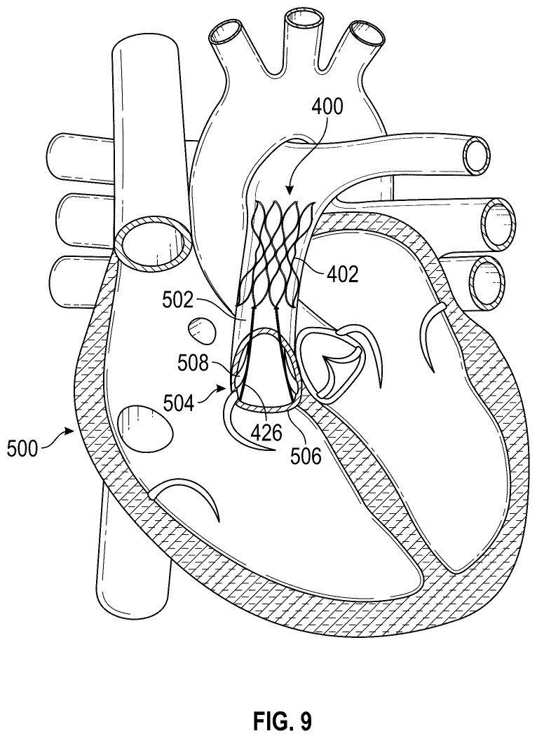

As shown in FIGS. 8A-9, the frame 400 also comprises one or more retention members 426 extending axially from the main body 402. The retention members 426 can each comprise a strut portion 428 that extends from an end portion (such as the first end portion 404) of the frame body, and a connector portion 430. The connector portions 430 are configured to releasably engage a corresponding connecting component of a delivery apparatus (such as frame connector 320 of delivery apparatus 300 disclosed herein). The geometry of the connector portions 430 can be selected to fit within one or more features of the corresponding connecting component when the frame 400 is connected to the delivery apparatus, and to come free of the one or more features of the corresponding connecting component when the frame 400 is released from the delivery apparatus. In some examples, the connector portions 430 can also engage the tissues of the patient's vasculature when the frame is implanted in a patient and in a deployed (that is, radially expanded) state, and to hold the frame 400 in place relative to a portion of the native vasculature. In the illustrated example, the connector portions 430 may be triangular or wedge-shaped, but it is to be understood that the connector portions 430 may have other geometries, such as square, rounded, or loop geometries so long as they are suitable for releasably engaging the corresponding features of the connecting component of the delivery apparatus.

The length of the retention members 426, and particularly the length L1 (shown in FIG. 8A) of the axially extending struts 428, can be selected based on the desired implantation location for the main body 402 relative to the native valve being replaced. It will be appreciated that, for example, the axially extending struts 428 may be shorter when the desired implantation location for the main body 402 is closer to the native valve, and longer when the desired implantation for the main body 402 is farther from the native valve. In some particular examples, the axially extending struts 428 can be 4-80 mm long, such as 8-80 mm, 12-80 mm, 16-80 mm, 20-80 mm, 24-80 mm, 28-40 mm, 32-80 mm, 36-80 mm, or 40-80 mm. In some examples, the axially extending struts 428 can be 4-76 mm, 4-72 mm, 4-68 mm, 4-64 mm, 4-60 mm, 4-56 mm, 4-52 mm, 4-48 mm, 4-44 mm, or 4-40 mm long. In specific examples, the axially extending struts 428 can be 4 mm, 8 mm, 12 mm, 16 mm, 20 mm, 24 mm, 28 mm, 32 mm, 36 mm, 40 mm, 44 mm, 48 mm, 52 mm, 56 mm, 60 mm, 64 mm, 68 mm, 72 mm, 76 mm, 80 mm long.

The length L1 of the axially extending struts 428 relative to the length of the main body 402 can vary. For example, the length of the axially extending struts can be within a range of 5-300% of the main body 402. In some examples, the length of the axially extending struts 428 can within a range of 5% to 200%, 5% to 190%, 5% to 180%, 5% to 170%, 5% to 160%, 5% to 150%, 5% to 140%, 5% to 130%, 5% to 120%, or 5% to 100% of the length of the main body 402. In some examples, the length of the axially extending struts 428 can range from 50% to 300%, 60% to 300%, 70% to 300%, 80% to 300%, 90% to 300%, or 100% to 300% of the length of the main body 402. In some specific examples, the axially extending struts 428 can be 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, or 300% of the length of the main body 402.

In some examples, as best illustrated in FIGS. 8A and 8D, the retention members 426 can extend from the major apices 422 of the major cells 416, such as those major cells 416 forming the first row 414a of cells 412 in the illustrated example. It is to be understood, however, that in other examples, the retention members 426 can extend from other portions of the main body 402, such as from the minor apices 424 of the minor cells 418, such as those forming the fifth row 414e of cells 412 in the illustrated example. It is also to be understood that in some examples, the retention members 426 can extend from other components of the frame body besides the apices 420, such as from the struts 410 or one or more valleys 432 circumferentially spaced between the apices 420 along an end portion 404, 406 of the frame body.

In some examples, such as the one illustrated in FIGS. 8A-8D, the frame 400 can comprise an equal number of major apices 422 and retention members 426 extending therefrom. In such examples, each apex 420 along the first end portion 404 of the main body 402 can have a retention member 426 extending therefrom, that is, there are no unattached or “free” apices 420 along the first end portion 404 of the main body 402. While examples of the frame 400 having no free apices 420 along the first end portion 404 of the main body 402 offer advantages in deployment and positioning of the frame, which are discussed in greater detail, below, it is to be understood that other examples of the frame 400 can have fewer retention members 426 than apices 420 along the first end portion 404 of the main body 402, such that the first end portion 404 comprises one or more apices 420 that are unattached to a retention member 426 (that is, are free apices 420).

When the frame 400 is implanted in the patient, the retention members 426 can extend through the annulus of the native heart valve while the main body 402 is axially spaced apart from the native heart valve. For example, as shown in FIG. 9, when the frame 400 is deployed in the heart 500 of a patient in the pulmonary artery 502, the frame body can be positioned upstream of and spaced apart from the native pulmonary valve 504.

The retention members 426 can extend through the annulus 506 of the native pulmonary valve 504. The retention members 426 may, in some examples, be shape set such that when the frame 400 is in the deployed (that is, the radially expanded) state, the retention members 426 press radially outwards against one or more leaflets 508 of the native pulmonary valve 504. As illustrated in FIG. 9, the leaflets 508 of the native pulmonary valve 504 can be held in an open state by the retention members 426, such that the native leaflets 508 are pinned against the wall of the pulmonary artery 502.

With continued reference to FIG. 9, the retention members 426 can engage with the leaflets 508, the native annulus 506, and/or the tissues of the pulmonary arteries. This fixes or secures the retention members 426, and therefore the frame 400, relative to the pulmonary valve 504, and prevents the main body 402 and any valvular structure secured therein from slipping or moving relative to the pulmonary valve 504.

While FIG. 9 depicts the frame 400 deployed in the pulmonary artery 502, downstream of the pulmonary valve 504, it is to be appreciated that the frame 400 can be installed in other positions or in other configurations. For example, the frame 400 could be deployed in the aorta, with the retention members deployed to secure the leaflets of the aortic valve in the open configuration and spacing the main body 402 downstream of the native aortic valve in the aorta. In other examples, the frame might be deployed adjacent to the mitral or tricuspid valve, with the retention members 426 pinning the respective leaflets of the valves in the open configuration and spacing the main body 402 apart from the respective valves.

As illustrated in FIGS. 8A-8D, the frame 400 can comprise a main body 402 that is substantially cylindrical, with a substantially uniform cross-section along the longitudinal axis of the main body 402.

In other examples, the frame 400 can have a tapered or hourglass cross-section, such as that discussed above in relation to the frame 100 and shown in FIGS. 1-4. In such examples, a relatively narrower middle portion (that is, a portion having a smaller diameter) of the frame can be positioned between the first end portion 404 and the second end portion 406 of the main body 402. In such examples, the narrow portion can function as a valve seat for securing a prosthetic heart valve, such as prosthetic heart valve 200 discussed above, and illustrated in FIG. 4. Advantageously this allows for prosthetic heart valves (such as prosthetic heart valve 200) with a variety of diameters to be positioned away from the native heart valve, according to the needs of the patient, while the retention members 426 pin the native leaflets open as previously described. Such a configuration of the frame 400 may be advantageous for implantation procedures to replace the pulmonary or mitral valves, because an hourglass shape allows the first end portion 404 and the second end portion 406 to engage with and secure to the vasculature of the patient, while allowing a smaller valve (such as a valve having a fully expanded diameter of about 29 mm) to be secured at the narrower middle portion of the frame.

In some examples, the frame 400 can be deployed as a docking stent. When deployed as a docking stent, the frame 400 can be configured to receive a prosthetic heart valve (such as the prosthetic heart valve 200 previously described and illustrated in FIG. 4). The prosthetic valve 200 can be implanted by first deploying the frame 400 at the implantation location and radially expanding the prosthetic heart valve to engage with the main body 402.

In other examples, such as that shown in FIG. 11 the frame 400 can, in lieu of the attached prosthetic heart valve previously described and illustrated in FIG. 4, have a valvular structure 600 directly attached to the frame. The valvular structure 600 can comprise a plurality of leaflets 602 (for example, 3 leaflets as illustrated in FIG. 11), configured to regulate the flow of blood through the main body 402 from the first end portion 404 to the second end portion 406, or from the second end portion 406 to the first end portion 404. In such examples, additional valvular structures such as skirts or sealing elements can also be attached to the frame 400.

The frame 400 can be deployed from a delivery apparatus, such the delivery apparatus 300, previously described, as well as those described in International Application No. PCT/US2022/018093. When used with the delivery apparatus 300, the frame 400 can be deployed in the same or substantially the same way as previously discussed in relation to the frame 100, and illustrated in FIGS. 6A through 6F, except for the differences described below.

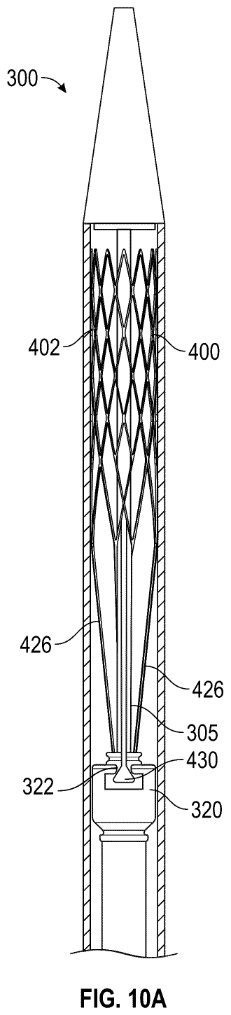

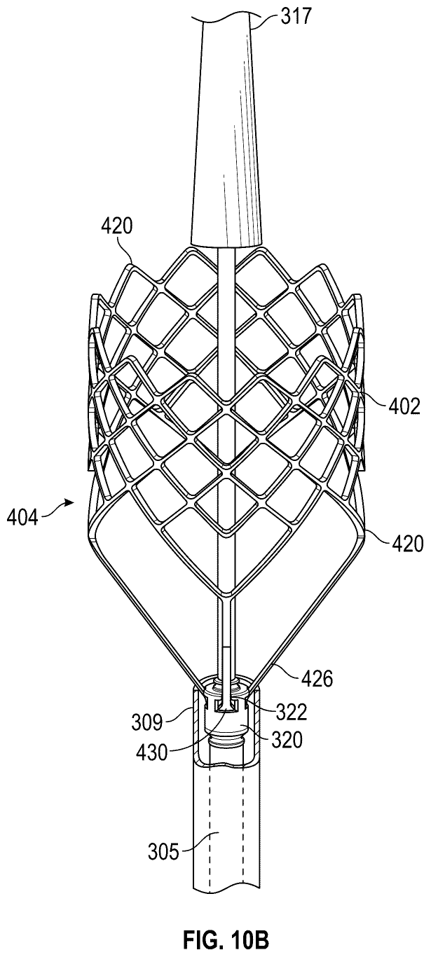

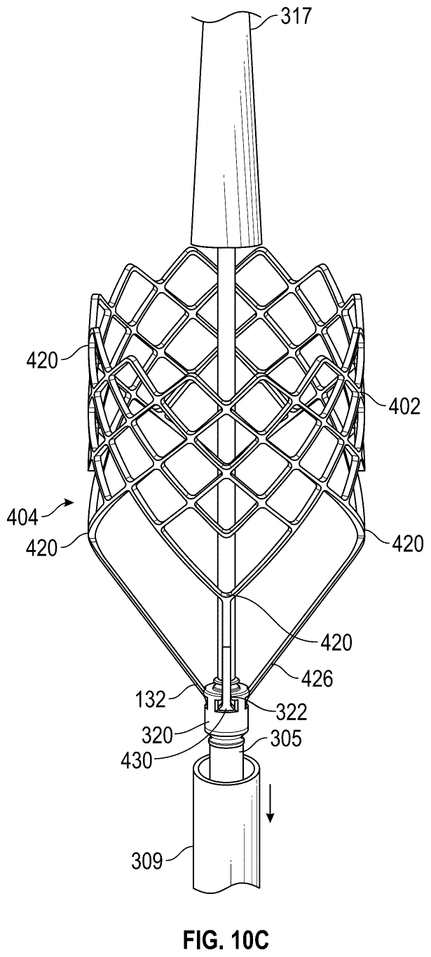

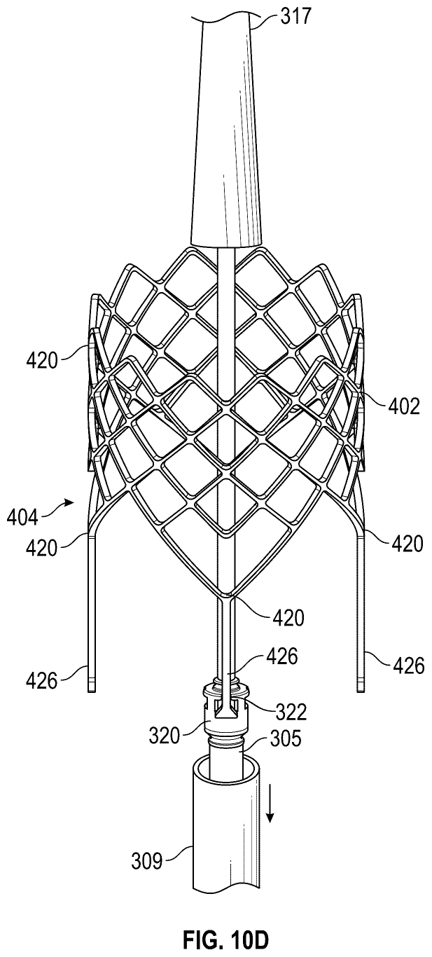

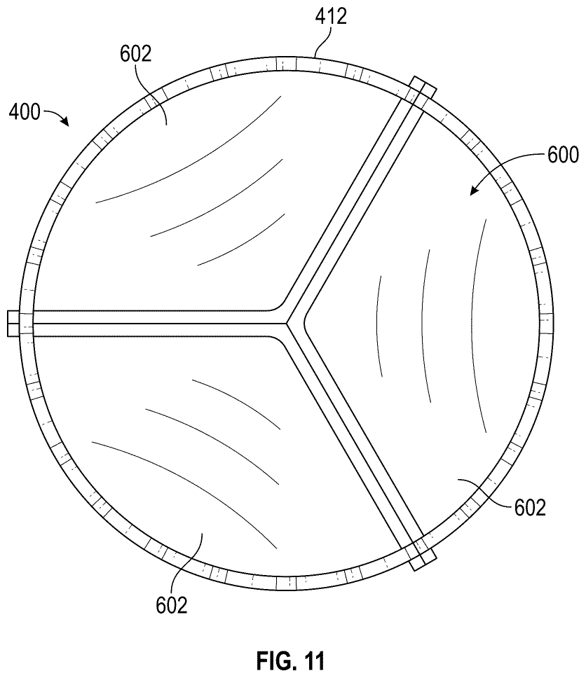

FIGS. 10A through 10D illustrate a method of deploying the frame 400 at a desired implantation location. Generally, the method comprises advancing the frame 400 in an undeployed state and contained within a delivery apparatus, such as the delivery apparatus 300 described above, to a desired delivery site and deploying the frame 400 at the desired implantation site.

As shown in FIG. 10A, the frame 400 can be disposed around a portion of the inner shaft 305, and proximal to the nosecone 317 of the delivery apparatus 300. The main body 402 is positioned towards the nosecone 317 of the delivery apparatus 300 and the retention members 426 oriented towards the proximal end of the delivery apparatus 300. The outer shaft 309 encloses the frame 400 and retains the frame 400 in a radially compressed state. The retention members 426 can be releasably coupled to the frame connector 320. In one example, the frame connector 320 can include one or more recesses 322 that receive a portion of one or more retention members 426, such as the connector portions 430 of the retention members 426.

The delivery apparatus 300 with the frame 400 in the undeployed state is advanced through the vasculature of the patient to the delivery location in substantially the same way as described above in relation to the deployment of the frame 100. At the implantation location, the outer shaft 309 is retracted (that is, moved in the proximal direction away from the nosecone 317) to expose the frame 400. Because the main body 402 is positioned closest to the nosecone 317, it is the first portion of the frame to be exposed by the retraction of the outer shaft 309. As each portion of the main body 402 is exposed by the retraction of the outer shaft 309, it expands radially, in the same or substantially the same way as described above and illustrated in FIG. 6C, gradually emerging from the outer shaft 309 and gradually expanding from a radially compressed state to a radially expanded state.

As the outer shaft 309 is retracted further, more of frame 400 is deployed from the delivery apparatus 300. As shown in FIG. 10B, the outer shaft 309 is retraced distally past the first end portion 404 of the main body 402 to advance the frame 400 from the undeployed state to a partially deployed state. This fully exposes the main body 402, while leaving at least some portion of the retention members 426 secured by the outer shaft 309. In this partially deployed state, the main body 402 may be allowed to radially expand to a fully expanded state.

Because the retention of members 426 remain at least partially enclosed within the outer shaft 309 while the frame 400 is in a partially deployed state, the portions of the retention members 426 that remain within the recesses 322, such as the connector portions 430, cannot exit the recesses 322. Thus, the retention members 426 remain secured to the frame connector 320, even after the main body 402 is fully exposed and radially expanded.

When the main body 402 is in the fully radially expanded state, the main body 402 may engage the native vasculature of the patient. Furthermore, any valvular structure, such as valvular structure 600 described above, or prosthetic heart valve, such as prosthetic heart valve 200 described above, may also be partially or fully deployed. When the prosthetic heart valve 200 or the valvular structure 600 is partially or fully deployed, the components (that is, the leaflets) of the prosthetic heart valve 200 or the valvular structure 600 can engage the flow of blood through the patient's vasculature. Advantageously, this allows an operating physician to evaluate the placement and performance of the frame 400 and any attached valvular structure or prosthetic heart valve within the vasculature of the patient before the frame 400 is fully detached from the delivery apparatus 300. For example, the physician can monitor one or more operational parameters of the valve, such as hemodynamic behavior or the pressure gradient across the valve before the frame 400 is fully detached from the delivery apparatus 300.

When the frame 400 is in the partially deployed state (that is, while the retention members 426 are still attached to the frame connector 320), it is possible to withdraw the frame connector 320 in a proximal direction, or to advance the outer shaft 309 in the distal direction, to bring the first end portion 404 of the frame body and a leading edge 324 of the outer shaft closer together until the main body 402 is re-enclosed within the outer shaft 309. As the frame 400 is re-enclosed within the outer shaft 309, the retention members 426 may assist in guiding the main body 402 into the outer shaft 309.

More particularly, as the frame 400 is withdrawn or re-enclosed in the outer shaft 309, the retention members 426 are drawn radially inwards. In turn the retention members 426 apply a radially inward force on the first end portion 404 of the main body 402. This draws the apices 420 along the first end portion 404 radially inwards, radially compressing the first end portion 404 of the main body 402 as main body 402 and the leading edge 324 of the outer shaft 309 draw closer together.

As the retraction of the frame 400 or the advancement of the outer shaft 309 over the frame 400 continues, the retention members 426 continue to guide the apices 420 of the first end portion 404 of the frame body radially inwards, until the leading edge 324 of the outer shaft 309 extends over the first end portion 404 of the frame body. Advantageously, in examples of frame 400 having no free apices 420 along the first end portion 404 of the main body 402, each apex 420 along the first end portion 404 of the main body 402 will be drawn radially inwards. This can prevent the apices 420 of the first end portion from catching on the leading edge 324 of the outer shaft 309 of the delivery apparatus 300 and facilitate repositioning and redeployment of the frame 400 at the desired implantation location.

Once the first end portion 404 of the main body 402, and particularly the apices 420 of the first row 414a of cells 412 have been withdrawn into the outer shaft 309, continued travel of the main body 402 into the outer shaft 309 is facilitated by the portions of the main body 402 already contained within the outer shaft 309. The main body 402 continues to radially compress as a greater portion of the main body 402 is drawn into the outer shaft 309, until the frame 400 is fully recovered and enclosed within the outer shaft 309 of the delivery apparatus 300.

In this way, the frame 400 can be recovered from the partially deployed state to the undeployed state so long as the retention members 426 are not decoupled from the frame connector 320. This may be of particular use if adjustments need to be made to the positioning of the frame 400 within the vasculature of the patient after the initial deployment of the main body 402 from the delivery apparatus 300.

Once no further adjustments to the position of the frame 400 within the vasculature of the patient are needed, the frame 400 can be fully deployed by retracting the outer shaft 309 (that is, moving the outer shaft 309 in the proximal direction) further. As shown in FIG. 10C for illustrative purposes, the outer shaft 309 can be retracted until the ends of the retention members 426, such as the connector portions 430 are fully exposed, at which point the frame 400 begins to fully deploy from the delivery apparatus 300.

As shown in FIG. 10D, when the outer shaft 309 is sufficiently retracted to fully expose the retention members 426, the retention members 426 disengage from the frame connector 320 and expand radially outwards to bring the frame 400 to the fully deployed state at the desired implantation site. Once the retention members 426 disengage from the frame connector 320, they radially expand to a fully deployed state. This allows the retention members 426 to engage the native vasculature, as shown in FIG. 9. Generally, the frame 400 can be positioned such that the retention members 426 extend through the annulus 506 of a native heart valve 504. Thus, as the retention members 426 radially expand, they engage the leaflets of the native heart valve (for example, the leaflets 508 of the native heart valve 504) as illustrated in FIG. 9.

In circumstances where the frame 400 is deployed as a docking stent with no valvular structure attached, a prosthetic heart valve can then be implanted in the radially expanded frame 400. The prosthetic heart valve can be advanced to a position within the main body 402, radially expanded within the main body 402, and then secured in place in the same or substantially the same way previously described regarding prosthetic heart valve 200 and shown in FIG. 4.

In this way, the example frames for a prosthetic heart valve disclosed herein can be positioned at a desired implantation site, such that the leaflets of the native heart valve upstream or downstream of the frame can be secured in the open configuration by the retention members. Simultaneously, the frame can be positioned downstream or upstream of the native heart valve and spaced apart from the native heart valve by a distance determined in part by the length of the retention members.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. A trial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples heat/thermal sterilization include steam sterilization and autoclaving. Examples of radiation for use in sterilization include, without limitation, gamma radiation, ultra-violet radiation and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the many possible examples to which the principles of the disclosure may be applied, it should be recognized that the illustrated examples are only preferred examples of the principles of the disclosure and should not be taken as limiting the scope of the disclosure. Rather, the scope of the disclosure is defined by the following claims. We therefore claim as our principles of disclosure all that comes within the scope and spirit of these claims.
Example 1. A prosthetic heart valve, comprising a radially expandable main body having a first end portion, a second end portion, and a plurality of interconnected struts, wherein the interconnected struts extend from the first end portion to the second end portion and defining a plurality of cells arranged in rows of cells; a valvular structure disposed within and attached to the radially expandable main body, configured to regulate a flow of blood through the radially expandable main body in one direction; and a plurality of retention members extending axially from the first end portion of the radially expandable main body; wherein the plurality of cells comprises a plurality of major cells and a plurality of minor cells, the minor cells being smaller than the major cells, wherein a row of cells nearest the first end portion of the radially expandable main body comprises major cells, and wherein the retention members extend axially from the major cells.
Example 2. The prosthetic heart valve of any example herein, particularly example 1, wherein first end portion is an inflow end portion and the second end portion is an outflow end portion.
Example 3. The prosthetic heart valve of any example herein, particularly example 1, wherein the first end portion is an outflow end portion and the second end portion is an inflow end portion.
Example 4. The prosthetic heart valve of any example herein, particularly examples 2-3, wherein the valvular structure is configured to regulate the flow of blood from the inflow end portion of the radially expandable main body to the outflow end portion of the radially expandable main body.
Example 5. The prosthetic heart valve of any example herein, particularly examples 1-4, wherein the radially expandable main body comprises 2-12 rows of cells.
Example 6. The prosthetic heart valve of any example herein, particularly examples 1-5, wherein the radially expandable main body comprises 5 rows of cells.
Example 7. The prosthetic heart valve of any example herein, particularly examples 1-6, wherein the major cells have a teardrop or heart shaped geometry.
Example 8. The prosthetic heart valve of any example herein, particularly examples 1-7, wherein the minor cells have a diamond shaped geometry.
Example 9. The prosthetic heart valve of any example herein, particularly examples 1-8 wherein a row of cells nearest to the second end portion of the radially expandable main body comprises minor cells.
Example 10. The prosthetic heart valve of any example herein, particularly examples 1-8, wherein a row of cells nearest to the second end portion of the radially expandable main body comprises major cells.
Example 11. The prosthetic heart valve of any example herein, particularly examples 1-10, wherein the first end portion of the radially expandable main body comprises 2-6 apices.
Example 12. The prosthetic heart valve of any example herein, particularly example 11, wherein the first end portion comprises 4 apices.
Example 13. The prosthetic heart valve of any example herein, particularly examples 1-12, wherein the second end portion of the radially expandable main body comprises 6-18 apices.
Example 14. The prosthetic heart valve of any example herein, particularly example 13, wherein the second end portion of the radially expandable main body comprises 12 apices.
Example 15. The prosthetic heart valve of any example herein, particularly examples 1-14, wherein the retention members comprise a first end portion attached to the radially expandable main body, and a second end portion terminating in a connector portion.
Example 16. The prosthetic heart valve of any example herein, particularly example 15, wherein the connector portion is configured to engage with tissues of a patient's vasculature.
Example 17. The prosthetic heart valve of any example herein, particularly examples 15-16, wherein the connector portion is triangular or wedge shaped.
Example 18. The prosthetic heart valve of any example herein, particularly examples 1-17, wherein the retention members are configured to extend through an annulus of a native heart valve when the prosthetic heart valve is deployed in a patient's vasculature.
Example 19. The prosthetic heart valve of any example herein, particularly examples 1-18, wherein the retention members are configured to retain one or more leaflets of a native heart valve in an open state when the prosthetic heart valve is deployed in a patient's vasculature.
Example 20. The prosthetic heart valve of any example herein, particularly examples 1-19, wherein the retention members are configured to self-expand to a radially expanded state when not radially constrained.
Example 21. The prosthetic heart valve of any example herein, particularly examples 1-20, wherein the retention members extend from the apices of the first end portion of the radially expandable main body.
Example 22. The prosthetic heart valve of any example herein, particularly examples 1-21, wherein each apex of the first end portion of the radially expandable main body has a retention member extending therefrom.
Example 23. The prosthetic heart valve of any example herein, particularly examples 1-22, wherein the retention members are 4-80 mm long.
Example 24. The prosthetic heart valve of any example herein, particularly example 23, wherein the retention members are 20-60 mm long.
Example 25. The prosthetic heart valve of any example herein, particularly examples 1-24, wherein the radially expandable main body has an axial length, and the retention members have a length that is 5%-300% of the axial length of the radially expandable main body.
Example 26. The prosthetic heart valve of any example herein, particularly examples 1-25, wherein the radially expandable main body is cylindrical, with a uniform or substantially uniform cross-section along a longitudinal axis of the radially expandable main body.
Example 27. The prosthetic heart valve of any example herein, particularly examples 1-25, wherein the radially expandable main body has an hourglass shape, with a middle portion between the first end portion and the second end portion, the middle portion having a smaller diameter than either the first end portion or the second end portion.
Example 28. The prosthetic heart valve of any example herein, particularly examples 1-27, further comprising a skirt or sealing element attached to the radially expandable main body.
Example 29. The prosthetic heart valve of any example herein, particularly examples 1-28, wherein the prosthetic heart valve is configured to be releasably attached to a delivery apparatus.
Example 30. The prosthetic heart valve of any example herein, particularly example 29, wherein the radially expandable main body can be deployed from the delivery apparatus without deploying the retention members from the delivery apparatus.
Example 31. The prosthetic heart valve of any example herein, particularly examples 29-30, wherein the radially expandable main body can be recovered into the delivery apparatus.
Example 32. An expandable frame, comprising a main body having a first end portion, a second end portion, and a longitudinal axis extending between the first end portion, and a plurality of interconnected struts extending from the first end portion to the second end portion along the longitudinal axis and forming a plurality of cells; and a plurality of retention members extending longitudinally from the first end portion; wherein the plurality of retention members is configured to extend through an annulus of a native heart valve and to retain one or more native leaflets of the native heart valve in an open state, and wherein the main body is configured to receive a prosthetic heart valve and secure the prosthetic heart valve at a location spaced apart longitudinally from the native heart valve.
Example 33. The expandable frame of any example herein, particularly example 32, wherein the first end portion is an inflow end portion and the second end portion is an outflow end portion.
Example 34. The expandable frame of any example herein, particularly example 32, wherein the first end portion is an outflow end portion and the second end portion is an inflow end portion.
Example 35. The expandable frame of any example herein, particularly examples 32-34, wherein the main body is configured to receive a prosthetic heart valve radially inwards of the main body, the prosthetic heart valve comprising a radially expandable frame securable to the main body and a valvular structure configured to regulate a flow of blood through the radially expandable frame.
Example 36. The expandable frame of any example herein, particularly examples 32-34, further comprising a valvular structure attached to the main body and configured to regulate a flow of blood through the main body.
Example 37. The expandable frame of any example herein, particularly example 36, further comprising a skirt or sealing element attached to the main body.
Example 38. The expandable frame of any example herein, particularly examples 32-37, wherein the expandable frame is self-expanding, and is configured to deploy to a radially expanded state when not radially constrained.
Example 39. The expandable frame of any example herein, particularly example 38, wherein the expandable frame comprises a shape memory material.
Example 40. The expandable frame of any example herein, particularly example 39, wherein the expandable frame comprises nitinol.
Example 41. The expandable frame of any example herein, particularly examples 32-40, wherein the retention members are configured to engage native tissues of a patient's vasculature when the expandable frame is in a radially expanded state.
Example 42. The expandable frame of any example herein, particularly examples 32-41, wherein the first end portion of the main body comprises a plurality of cell apices, and the retention members extend from the plurality of cell apices.
Example 43. The expandable frame of any example herein, particularly example 42, wherein a retention member extends from each cell apex of the first end portion of the main body.
Example 44. The expandable frame of any example herein, particularly examples 42-43, wherein the first end portion of the main body comprises 2-6 cell apices.
Example 45. The expandable frame of any example herein, particularly examples 42-44, wherein the second end portion of the main body comprises 6-18 cell apices.
Example 46. The expandable frame of any example herein, particularly examples 32-45, wherein the plurality of cells comprises a plurality of major cells and a plurality of minor cells, wherein the major cells are larger than the minor cells, and wherein plurality of cells forms rows of cells between the first end portion and the second end portion of the main body.
Example 47. The expandable frame of any example herein, particularly example 46, wherein the row of cells nearest the first end portion of the main body comprises exclusively major cells.
Example 48. The expandable frame of any example herein, particularly examples 46-47, wherein the main body comprises 2-12 rows of cells.
Example 49. The expandable frame of any example herein, particularly example 48, wherein the main body comprises 4-6 rows of cells.
Example 50. The expandable frame of any example herein, particularly examples 32-49, wherein the retention members are 4-80 mm long.
Example 51. The expandable frame of any example herein, particularly examples 32-49, wherein the retention members are between 5% and 300% of a length of the main body along the longitudinal axis.
Example 52. The expandable frame of any example herein, particularly examples 32-51, wherein the main body is cylindrical with a substantially uniform cross-section along the longitudinal axis.
Example 53. The expandable frame of any example herein, particularly examples 32-51, wherein the main body is hourglass shaped, with a middle portion between the first end portion and the second end portion, and where a diameter of the main body at the middle portion is smaller than a diameter of the main body at the first end portion or the second end portion.
Example 54. The expandable frame of any example herein, particularly examples 32-53, wherein the expandable frame is configured to be releasably attached to a delivery apparatus.
Example 55. The expandable frame of any example herein, particularly example 54, wherein the main body can be deployed from the delivery apparatus without deploying the retention members from the delivery apparatus.
Example 56. The expandable frame of any example herein, particularly examples 54-55, wherein the main body can be recovered into the delivery apparatus.
Example 57. A medical assembly, comprising a first frame comprising a main body having a first end portion, a second end portion, and a longitudinal axis extending between the first end portion and the second end portion and, a plurality of interconnected struts extending from the first end portion to the second end portion and forming a plurality of cells, and a plurality of retention members extending longitudinally from the first end portion of the main body; and a prosthetic heart valve disposed within and secured to the main body, the prosthetic heart valve comprising a radially expandable second frame and a valvular structure disposed within the radially expandable second frame and configured to regulate a flow of blood through the prosthetic heart valve; wherein the plurality of retention members is configured to extend through an annulus of a native heart valve and to retain one or more native leaflets of the native heart valve in an open state, and wherein the main body is configured to secure the prosthetic heart valve at a location spaced apart longitudinally from the native heart valve.
Example 58. The medical assembly of any example herein, particularly example 57, wherein the retention members are between 4 and 80 mm long.
Example 59. The medical assembly of any example herein, particularly example 57, wherein the main body has a length along the longitudinal axis, and the length of the retention members is 5%-300% of the length of the main body.
Example 60. The medical assembly of any example herein, particularly examples 57-59, wherein the plurality of cells of the main body comprises a plurality of major cells and a plurality of minor cells, wherein the major cells are larger than the minor cells.
Example 61. The medical assembly of any example herein, particularly examples 57-60, wherein the first end portion and the second end portion of the main body comprise a plurality of apices formed by an intersection of interconnected struts.
Example 62. The medical assembly of any example herein, particularly example 61, wherein the first end portion of the main body comprises 2-6 apices.
Example 63. The medical assembly of any example herein, particularly examples 61-62, wherein each apex of the first end portion of the main body has a retention member extending therefrom.
Example 64. The medical assembly of any example herein, particularly examples 57-63, wherein the frame is configured to be releasably attached to a delivery apparatus.
Example 65. The medical assembly of any example herein, particularly example 64, wherein the main body can be deployed from the delivery apparatus without deploying the retention members from the delivery apparatus.
Example 66. The medical assembly of any example herein, particularly examples 64-65, wherein the main body can be recovered into the delivery apparatus.
Example 67. A method comprising advancing a delivery apparatus comprising an outer shaft containing a prosthetic heart valve, the prosthetic heart valve comprising a frame having a main body and a plurality of retention members extending axially from the main body, in a distal direction through a vasculature of a patient to a native annulus; partially deploying the prosthetic heart valve from the delivery apparatus adjacent to a native heart valve of the patient, wherein when the prosthetic heart valve is partially deployed, a valvular structure is engaged with a flow of blood and the retention members are attached to the delivery apparatus; advancing or retracting the deployed prosthetic heart valve in a direction defined by the flow of blood through the native heart valve until the retention members extend through a native annulus of the native heart valve; detaching the prosthetic heart valve from the delivery apparatus; and expanding the retention members to a fully expanded state, wherein when the retention member are in the fully expanded state, the retention members contact one or more native leaflets of the native heart valve and retain the one or more native leaflets in an open state; wherein the prosthetic heart valve further comprises a valvular structure disposed within the frame and configured to regulate the flow of blood through the frame, wherein the prosthetic heart valve is secured to the delivery apparatus by the plurality of axially extending retention members, and wherein the valvular structure is axially spaced apart from the native annulus of the native heart valve in the direction defined by the flow of blood.
Example 68. The method of any example herein, particularly example 67, wherein partially deploying the prosthetic heart valve from the delivery apparatus comprises retracting the outer shaft in a proximal to expose the main body of the frame.
Example 69. The method of any example herein, particularly examples 67-68, wherein detaching the prosthetic heart valve from the delivery apparatus comprises retracting the outer shaft to fully expose the retention members, thereby releasing them from the delivery apparatus.
Example 70. The method of any example herein, particularly examples 67-69, wherein the prosthetic heart valve is deployed in an aorta, with the retention members extending through the native annulus of an aortic valve.
Example 71. The method of any example herein, particularly examples 67-69, wherein the prosthetic heart valve is deployed to replace a mitral valve, with the retention members extending through the native annulus of the mitral valve.
Example 72. The method of any example herein, particularly examples 67-69, wherein the prosthetic heart valve is deployed to replace a tricuspid valve, with the retention members extending through the native annulus of the tricuspid valve.
Example 73. The method of any example herein, particularly examples 67-69, wherein the prosthetic heart valve is deployed to replace a pulmonary valve, with the retention members extending through the native annulus of the pulmonary valve.
Example 74. The method of any example herein, particularly examples 67-73, wherein the method further comprises analyzing a position of the prosthetic heart valve after the prosthetic heart valve is partially deployed or analyzing one or more operational parameters of the valve after the prosthetic heart valve is partially deployed.
Example 75. The method of any example herein, particularly example 74, wherein the operational parameters include one or more of hemodynamic behavior or pressure gradients.
Example 76. The method of any example herein, particularly examples 74-75, wherein the method further comprises adjusting a placement of the prosthetic heart valve.
Example 77. The method of any example herein, particularly examples 68-76, wherein the method further comprises recapturing the prosthetic heart valve within the delivery apparatus.
Example 78. The method of any example herein, particularly example 77, wherein recapturing the prosthetic heart valve within the delivery apparatus comprises advancing the outer shaft in a distal direction or retracting the prosthetic heart valve in a proximal direction until the outer shaft at least partially encloses the main body of the frame.
Example 79. The method of any example herein, particularly example 78, further comprising redeploying the prosthetic heart valve from the delivery apparatus by retracting the outer shaft in the proximal direction until at least the main body of the frame is exposed.
Example 80. The method of any example herein, particularly examples 68-79, wherein the main body of the frame comprises a first end portion and a second end portion, the retention members extend axially from the first end portion of the main body of the frame, and the first end portion and second end portion each comprise a plurality of apices formed by an intersection of interconnected struts.
Example 81. The method of any example herein, particularly example 80, wherein a first apex of the first end portion of the main body has a first retention member extending therefrom.
Example 82. The method of any example herein, particularly example 81, wherein when the prosthetic heart valve is recaptured within the delivery apparatus, the first retention member guides the first apex of the first end portion of the main body into the outer shaft of the delivery apparatus.
Example 83. The method of any example herein, particularly examples 68-82, wherein the prosthetic heart valve comprises three or more retention members.
Example 84. The method of any example herein, particularly examples 68-83, wherein the first end portion of the main body comprises fewer apices than the second end portion of the main body.
Example 85. A method comprising advancing a delivery apparatus comprising an outer shaft containing a radially expandable frame, the radially expandable frame comprising a main body and a plurality of retention members extending axially from the main body, through a vasculature of a patient to a native annulus; partially deploying the radially expandable frame from the delivery apparatus adjacent to a native heart valve of the patient; positioning the radially expandable frame relative to the native heart valve such that the retention members extend through a native annulus of the native heart valve; fully deploying the radially expandable frame from the delivery apparatus; expanding the radially expandable frame to a fully expanded state, wherein an outer diameter of the main body contacts the vasculature of the patient and the retention members contact one or more native leaflets of the native heart valve and retain the one or more native leaflets in an open state; wherein when the radially expandable frame is fully radially expanded, the main body is axially spaced apart from the native heart valve, and wherein the radially expandable frame is configured to receive a prosthetic heart valve and secure the prosthetic heart valve relative to the native heart valve.
Example 86. The method of any example herein, particularly example 85, wherein the radially expandable frame is deployed upstream of the native heart valve.
Example 87. The method of any example herein, particularly example 85, wherein the radially expandable frame is deployed downstream of the native heart valve.
Example 88. The method of any example herein, particularly examples 85-87, wherein the retention members each comprise an axially extending strut connected to the main body and a connector portion connected to the axially extending strut.
Example 89. The method of any example herein, particularly example 88, wherein the axially extending struts of the retention members are configured to retain the native leaflets in the open state by securing the native leaflets between the vasculature of the patient and the axially extending strut when the radially expandable frame is fully deployed.
Example 90. The method of any example herein, particularly examples 88-89, wherein the connector portion is configured to engage the vasculature of the patient when the radially expandable frame is fully deployed.
Example 91. The method of any example herein, particularly examples 85-90, further comprising advancing a prosthetic heart valve through the vasculature of the patient and securing the prosthetic heart valve to the radially expandable frame.
Example 92. The method of any example herein, particularly examples 85-90, wherein the prosthetic heart valve is spaced apart from the native heart valve in an upstream direction or a downstream direction.
Example 93. The method of any example herein, particularly examples 85-92, wherein partially deploying the prosthetic heart valve from the delivery apparatus comprises retracting the outer shaft in a proximal direction to expose the main body, thereby allowing the main body to deploy to a radially expanded state while the retention members are still retained by the outer shaft.
Example 94. The method of any example herein, particularly example 93, wherein fully deploying the prosthetic heart valve from the delivery apparatus comprises retracting the outer shaft to expose the retention members, thereby allowing the retention members to detach from the delivery apparatus and deploy to a radially expanded state.
Example 95. The method of any example herein, particularly examples 85-94, further comprising recapturing the radially expandable frame from a partially deployed state to an undeployed state.
Example 96. The method of any example herein, particularly example 95, wherein recapturing the radially expandable frame comprises advancing the outer shaft in a distal direction until the outer shaft at least partially encloses the main body.
Example 97. The method of any example herein, particularly example 95, wherein recapturing the radially expandable frame comprises retracting the radially expandable frame in a proximal direction until the outer shaft at least partially encloses the main body.
Example 98. The method of any example herein, particularly examples 85-97, wherein the main body has a first end portion and a second end portion, the first end portion and the second end portion comprise a plurality of apices formed by an intersection of a plurality of interconnected struts, and the retention members extend from the first end portion of the main body.
Example 99. The method of any example herein, particularly example 98, wherein the first end portion of the main body comprises 2-6 apices.
Example 100. The method of any example herein, particularly examples 98-99, wherein each apex of the first end portion of the main body has a retention member extending therefrom.
Example 101. The method of any example herein, particularly examples 85-100, wherein the main body has an axial length and the retention members have a length that is 5%-300% of the axial length of the main body.
Example 102. A method comprising advancing a delivery apparatus comprising an outer shaft containing a radially expandable frame, the radially expandable frame comprising a main body and a plurality of retention members extending axially from the main body, through a vasculature of a patient to a native annulus; partially deploying the radially expandable frame from the delivery apparatus adjacent to a native heart valve of the patient; recapturing the radially expandable frame within the delivery apparatus; adjusting the position of the radially expandable frame relative to the native heart valve of the patient; and fully deploying the radially expandable frame from the delivery apparatus such that the radially expandable frame engages the vasculature of the patient and the plurality of retention members disengage from the delivery apparatus.
Example 103. A method comprising sterilizing the medical assembly, the radially expandable frame, the main body, or the prosthetic heart valve of any example herein.

1. An expandable frame comprising:
a main body having a first end portion, a second end portion, a longitudinal axis extending between the first end portion and the second end portion, and a plurality of interconnected struts extending from the first end portion to the second end portion along the longitudinal axis and forming a plurality of cells; and
a plurality of retention members extending longitudinally from the first end portion,
wherein the plurality of retention members is configured to extend through an annulus of a native heart valve and retain one or more native leaflets of the native heart valve in an open state, and
wherein the main body is configured to receive a prosthetic heart valve and secure the prosthetic heart valve at a location spaced apart longitudinally from the native heart valve.

2. The expandable frame of claim 1, wherein the first end portion is an inflow end portion and the second end portion is an outflow end portion.

3. The expandable frame of claim 1, wherein the first end portion is an outflow end portion and the second end portion is an inflow end portion.

4. The expandable frame of claim 1, wherein the main body is configured to receive the prosthetic heart valve radially inwards of the main body, and wherein the prosthetic heart valve comprises a radially expandable frame securable to the main body and a valvular structure configured to regulate a flow of blood through the radially expandable frame.

5. The expandable frame of claim 1, further comprising a valvular structure attached to the main body and configured to regulate a flow of blood through the main body.

6. The expandable frame of claim 5, further comprising a skirt or sealing element attached to the main body.

7. A medical assembly comprising:
a first frame comprising a main body having a first end portion, a second end portion, a longitudinal axis extending between the first end portion and the second end portion, a plurality of interconnected struts extending from the first end portion to the second end portion and forming a plurality of cells, and a plurality of retention members extending longitudinally from the first end portion of the main body; and
a prosthetic heart valve disposed within and secured to the main body, the prosthetic heart valve comprising a radially expandable second frame and a valvular structure disposed within the radially expandable second frame and configured to regulate a flow of blood through the prosthetic heart valve,
wherein the plurality of retention members is configured to extend through an annulus of a native heart valve and retain one or more native leaflets of the native heart valve in an open state, and
wherein the main body is configured to secure the prosthetic heart valve at a location spaced apart longitudinally from the native heart valve.

8. The medical assembly of claim 7, wherein the retention members are between 4 and 80 mm long.

9. The medical assembly of claim 7, wherein the main body has a length along the longitudinal axis, and the length of the retention members is 5%-300% of the length of the main body.

10. The medical assembly of claim 7, wherein the plurality of cells of the main body comprises a plurality of major cells and a plurality of minor cells, and wherein the major cells are larger than the minor cells.

11. The medical assembly of claim 7, wherein the first end portion and the second end portion of the main body comprise a plurality of apices formed by an intersection of interconnected struts.

12. The medical assembly of claim 11, wherein the first end portion of the main body comprises 2-6 apices.

13. The medical assembly of claim 11, wherein each apex of the first end portion of the main body has a retention member extending therefrom.

14. A method comprising:
advancing a delivery apparatus through a vasculature of a patient to a native annulus, wherein the delivery apparatus comprises an outer shaft containing a prosthetic heart valve, and wherein the prosthetic heart valve comprises a frame having a main body and a plurality of retention members extending axially from the main body;
partially deploying the prosthetic heart valve from the delivery apparatus adjacent to a native heart valve of the patient, wherein when the prosthetic heart valve is partially deployed, a valvular structure is engaged with a flow of blood and the retention members are attached to the delivery apparatus;
advancing or retracting the deployed prosthetic heart valve in a direction defined by the flow of blood through the native heart valve until the retention members extend through a native annulus of the native heart valve;
detaching the prosthetic heart valve from the delivery apparatus; and
expanding the retention members to a fully expanded state, wherein when the retention members are in the fully expanded state, the retention members contact one or more native leaflets of the native heart valve and retain the one or more native leaflets in an open state, wherein the prosthetic heart valve further comprises a valvular structure disposed within the frame and configured to regulate the flow of blood through the frame,
wherein the prosthetic heart valve is secured to the delivery apparatus by the plurality of axially extending retention members, and
wherein the valvular structure is axially spaced apart from the native annulus of the native heart valve in the direction defined by the flow of blood.

15. The method of claim 14, wherein partially deploying the prosthetic heart valve from the delivery apparatus comprises retracting the outer shaft to expose the main body of the frame.

16. The method of claim 14, wherein detaching the prosthetic heart valve from the delivery apparatus comprises retracting the outer shaft to fully expose the retention members, thereby releasing them from the delivery apparatus.

17. The method of claim 14, wherein the prosthetic heart valve is deployed in an aorta, with the retention members extending through the native annulus of an aortic valve.

18. The method of claim 14, wherein the prosthetic heart valve is deployed to replace a mitral valve, with the retention members extending through the native annulus of the mitral valve.

19. The method of claim 14, wherein the prosthetic heart valve is deployed to replace a tricuspid valve, with the retention members extending through the native annulus of the tricuspid valve.

20. The method of claim 14, wherein the prosthetic heart valve is deployed to replace a pulmonary valve, with the retention members extending through the native annulus of the pulmonary valve.
